(19) United States
(12) Patent Application Publication
(10) Pub. No.: US 2004/0081672 A1
(43) Pub. Date: Apr. 29, 2004

(54) Niacinamide, niacin, and niacin esters based delivery systems for treating topical disorders of skin and skin aging

(76) Inventor: Shyam K. Gupta, Scottsdale, AZ (US)

Correspondence Address: SHYAM K. GUPTA, BIODERM RESEARCH, 5221 E. WINDROSE DRIVE, SCOTTSDALE, AZ, 85254, US

(21) Appl. No.: 10/280,519

(22) Filed: Oct. 25, 2002

(51) Int. Cl. A61K 7/00; A61K 31/455; A61K 31/728; A61K 31/665; A61K 31/655

(52) U.S. Cl. 424/401; 514/356; 514/54; 514/101; 514/157; 514/18; 514/171; 514/33

(57) ABSTRACT

The present invention relates to application of niacinamide, niacin, and niacin esters (nicotinic acid esters) derivatives of skin beneficial organic acids (henceforth called “derivatives”) for the synergistic treatment or prevention of topical disorders of skin such as acne, rosacea, skin wrinkles, age-spots, canker sores, striae distensae (stretch marks), pimples, and skin redness. These “derivatives” are made by a very simple in-situ method. These “derivatives” provide enhanced bioavailability of both ingredients that are combined to form such derivatives. The “derivatives” of present invention can be formulated in a wide variety of delivery systems including traditional water and oil emulsions, suspensions, colloids, solutions, or anhydrous systems. Niacin and certain niacin ester derivatives prepared by the in-situ method are free of flushing effect (a warm feeling in the skin usually associated with redness and itching) normally experienced with niacin and certain niacin esters themselves.

### BACKGROUND OF THE INVENTION:

This invention relates to in-situ preparation of the derivatives of niacinamide, niacin, and niacin esters (nicotinic acid esters) with skin beneficial organic acids and their application in the synergistic treatment and regulation of topical disorders of skin such as skin aging, wrinkles, acne, rosacea, age-spots, canker sores, striae distensae (stretch marks), pimples, and skin redness. The in-situ method also permits the simple preparation of certain novel derivatives of niacinamide, niacin, and niacin esters from commonly available ingredients. Most niacin and niacin ester derivatives produced by the in-situ method do not show flushing effects (a warm feeling in the skin usually associated with redness and itching) that are traditionally experienced with niacin and certain niacin esters. The compositions that contain such derivatives of niacinamide, niacin, or niacin esters can be traditional water and oil emulsions, suspensions, colloids, solutions, or anhydrous systems.

### OBJECTS OF THE INVENTION:

This invention relates to in-situ preparation of the derivatives of niacinamide, niacin, and niacin esters (nicotinic acid esters) with skin beneficial organic acids, and their application in skin care compositions that provide synergistic treatment and regulation of topical disorders of skin such as skin aging, wrinkles, acne, rosacea, age-spots, canker sores, striae distensae (stretch marks), pimples, and redness.

This invention also relates to compositions that include derivatives of niacinamide, niacin, and niacin esters with skin beneficial organic acids that are prepared in-situ from the combination of niacinamide, niacin, or niacin esters with such organic acids.

In a further respect, this invention relates to niacinamide, niacin, and niacin ester derivatives of skin beneficial organic acids that provide enhanced bioavailability of both niacinamide, niacin or niacin ester and organic acid moieties thus combined to form such derivatives.

In a further respect, the invention relates to niacinamide, niacin, and niacin ester derivatives of organic acids that provide a combination of the skin beneficial properties of ingredients thus combined and additionally provide synergistic benefits. For example, niacinamide salicylate is made by the combination of niacinamide and salicylic acid. Niacinamide salicylate has the combination benefits of both niacinamide and salicylic acid in the treatment of acne. The absorption and penetration of niacinamide salicylate is more enhanced than the absorption of either niacinamide or salicylic acid, if used alone. Thus, niacinamide salicylate is more effective in treating acne due to its better synergistic absorption into the skin. In the same example if niacinamide lipoate is also used along with niacinamide salicylate then the acne treatment effect of niacinamide salicylate is further increased several-fold due to the synergistic effect of the present invention. In another example, hydroquinone is a drug ingredient approved by the FDA for skin whitening. The use of niacinamide ascorbate in addition to hydroquinone in a composition increases the skin whitening effect of hydroquinone. If niacinamide ascorbate and niacinamide lactate is also used in combination with hydroquinone, then the skin whitening effect of hydroquinone is further enhanced several-fold.

In a further respect, this invention relates to niacin or niacin ester derivatives that do not show flushing (a warm feeling in the skin usually associated with redness and itching).

In a further respect, this invention relates to in-situ preparation of novel derivatives of niacinamide, niacin, or niacin esters with skin beneficial organic acids that can be made either in anhydrous systems, solutions, or traditional water and oil emulsion systems, thus offering a wide choice of delivery systems.

### BRIEF DESCRIPTION OF THE INVENTION

I have discovered a simple in-situ preparation of the derivatives of niacinamide, niacin, and niacin esters with certain organic acids, and their application in topical cosmetic and pharmaceutical compositions that provide synergistic treatment and regulation of topical disorders of skin such as skin aging, wrinkles, acne, rosacea, age-spots, canker sores, striae distensae (stretch marks), pimples, and skin redness. Some of such in-situ prepared niacin derivatives do not show flushing effects (a warm feeling in the skin usually associated with redness and itching). Moreover, such derivatives of niacinamide, niacin, or niacin esters in a combination composition can be made in a stable topical formulation by the in-situ method from readily available starting materials. The in-situ method also permits the preparation of certain novel derivatives of niacinamide, niacin, and niacin esters with skin beneficial organic acids. The compositions made by the in-situ method possess the additional advantage that they can be made in anhydrous systems, solutions, or traditional water and oil emulsion systems, thus offering a wide choice of delivery systems.

### DETAILED DESCRIPTION OF THE INVENTION

Niacin, also known as vitamin B.sub.3, is the common name for nicotinic acid. The physiologically active form of niacin is niacinamide, also a member of the vitamin B.sub.3 family of compounds. Niacin and niacinamide (nicotinic acid amide) function in the body as components of two coenzymes: nicotinamide adenine dinucleotide (NAD) and nicotinamide adenine dinucleotide phosphate (NADP). Until recently, these vitamin B.sub.3 compounds were used exclusively to treat niacin deficiency and pellagra. Today, however, vitamin B.sub.3 compounds have also found use in the area of skin care actives. However, when applied to the skin in crystalline form (i.e., powder), vitamin B.sub.3 compounds tend to impart a rough feel to the skin. In the past, the crystalline vitamin B.sub.3 compounds were solubilized in a polar solvent before application to skin, thus alleviating the rough feel of the crystals. However, solubilization reduced the efficacy of the vitamin B.sub.3 compound upon contact with the skin. These problems have been discussed by Walling et al. (U.S. Pat. No. 6,455,055) in detail. Walling et al. also reported the need for cosmetic compositions comprising unsolubilized crystalline vitamin B.sub.3 compound(s) and discovered certain cosmetic formulations incorporating the crystalline vitamin B.sub.3 compounds of specific particle size in combination with an emollient. However, such formulations were only applicable to lipstick delivery system, and hence of very limited applicability in other cosmetic products. It is also difficult to obtain niacinamide or niacin that meet the strict particle size specifications mentioned by Walling et al. SaNogueira et al. (U.S. Pat. No. 6,174,533) discuss that while a variety of compounds have been described in the art as being useful for regulating fine lines, wrinkles, acne, pimples, and other forms of undesirable skin surface texture, niacinamide and niacin have shown most promise in regulating skin conditions including fine lines, wrinkles, uneven or rough surface, and photo-damaged skin. Sauermann et al. (U.S. Pat. No. 6,428,779) report the need for antiage compositions. According to these authors, skin ages as a result of endogenous, genetically determined influences. Exogenous factors, such as UV light and chemical noxae, can have a cumulative effect and accelerate the natural aging processes. This results in numerous degenerative processes that lead, depending on the extent of the influencing factors, inter alia, to the following structural changes and damage in the dermis and epidermis (e.g. also to dermatoheliosis):

a) Degeneration of the microvascular system.

b) Flaccidity and development of wrinkles, partly due to a decrease in and crosslinking of collagen, accumulation of glycosaminoglycan (base substance) and solar elastosis (elastin clumping).

c) Flattening of the retial cones. This is associated with the reduction in the area between the dermis and epidermis via which substances are exchanged for nutrition and purification of the epidermis.

d) Restricted regenerative turnover in the epidermis, associated with defective development of the horny layer (disturbed hornification), leading to drying out of the skin, to roughness of the skin and to chapping of the skin.

e) Defective regulation of cell division (proliferation) and cell maturation (differentiation) in the epidermis, which results in cellular atypia and atrophies and the loss in polarity.

f) Local hyper- and hypo pigmentation and abnormal pigmentation (age spots).

It would appear from the above biochemical problems related to skin disorders that high bioavailability of niacinamide and niacin compositions is very important, but it is not easily achieved due to limited solubility and stability of niacinamide and niacin in various cosmetic compositions. It would thus be of great importance to develop niacinamide and niacin derivatives that could circumvent the above problems. It would be additionally beneficial if such derivatives could provide enhanced bioavailability. It would be even more advantageous if such derivatives of niacinamide and niacin can be easily made from commonly available ingredients. Furthermore, if such derivatives could also provide synergistic efficacy, then truly innovative compositions can be developed to fill current high consumer demand for antiage skin care compositions.

Niacinamide and niacin, as members of Vitamin B3 group, are well known for their various skin beneficial properties. The combination of niacinamide or niacin with other skin beneficial ingredients has been used in the prior art to develop combinations with synergistic skin beneficial properties. For example, Jacobson et al. (U.S. Patent Application 20010049382 and 20020034482) describe niacin and certain synthetic esters of niacin that are useful for the treatment of hyperlipidemia and for improving the delivery of oxygen to cells. Horrobin et al (U.S. Pat. No. 6,015,821) describe the preparation of esters of nicotinic acid that are useful for the control of dermatological disorders, among other applications. It would be advantageous if such esters can be made into enhanced bioavailable forms that can also offer synergistic efficacy for the alleviation of targeted skin ailments. Niacinamide and niacin have been used for several skin beneficial formulations. For example, a commercial product called “Papulex” is a very good selling acne treatment product that contains 4% niacinamide in a gel form marketed by Euroderma, Surrey, England. Zhang et al. (U.S. Patent Application 20020106384) describe skin-whitening compositions that contain niacinamide. Robinson et al. (U.S. Pat. No. 6,444,647) describe a skin care composition that contains a combination of niacinamide and salicylic acid. However, this formulation must also contain farnesol and phytantriol as skin care actives for its efficacy. It is not clear if the skin beneficial effect is from the combination of niacinamide and farnesol or phytantriol, or a mixture of all the ingredients. Similarly, Bissett et al. (U.S. Pat. No. 6,183,761) describe a combination of niacinamide and salicylic acid for regulating skin disorders, but this composition must also include polycyclic compounds, some of which are anti-inflammatory in their property. It is thus not clear again if the efficacy of this formulation is actually from polycyclics, and not from niacinamide and salicylic acid. In the above cases, any preparation or application of niacinamide salicylate was not reported. It would thus be of interest to study if niacinamide salicylate would have synergistic benefit of both niacinamide and salicylic acid in the treatment of acne-related skin problems. Fitzjarrell (U.S. Pat. No. 6,432,430) has prepared an exfoliating scrub that contains niacinamide for skin beneficial effects. However, this product also contains harsh scrubbing agents that expose fresh cells to permit the absorption of niacinamide into the skin. This is both inconvenient and sometimes painful or can cause skin irritation when a consumer with sensitive skin has to scrub skin. It would be much more advantageous if a niacinamide formulation was easily absorbed, more bioavailable, and did not require scrubbing the skin with an exfoliating product. Another acne treatment spray composition has been claimed by Fitzjarrell (U.S. Pat. No. 5,989,523), but for optimum benefits an exfoliating scrub is recommended prior to the application of the niacinamide spray composition. U.S. Pat. No. 5,968,528 to Deckner et al. describes niacinamide compositions beneficial for skin compatibility. U.S. Pat. No. 5,962,482 to Bissett describes cellulite reduction with a niacinamide composition. The oily appearance of skin can be controlled by a niacinamide composition described in U.S. Pat. Nos. 5,980,921 and 5,833,998 to Biederman et al. U.S. Pat. Nos. 6,238,678 and 5,939,082 to Oblong et al. describe a niacinamide composition for the treatment of skin aging. It would thus be obvious if niacinamide compositions with enhanced absorption, better bioavailability, and synergistic skin care benefits could be developed that are easy to prepare, stable, and cosmetically appealing.

In prior the art, the preparation of such derivatives of niacinamide or niacin with organic acids has been very difficult, inconvenient, or even not possible and thus not reported. For example, Niacinamide Ascorbate, a salt formed by the reaction of niacinamide (an organic base) and ascorbic acid, has been possible only with special handling, as reported by C. W. Bailey et al., J Amer. Chem. Soc., 67, 1184-5, (1945). Similarly, Chitosan Niacinamide Ascorbate salt has been reported in U.S. Patent Application No. 20020058704 in a water solution from niacinamide and ascorbic acid, but this involves a two-step process whereby niacinamide ascorbate must first be prepared and then reacted with chitosan in a second chemical step. Moreover, the stability of chitosan niacinamide ascorbate in contact with water, or any synergistic skin, hair, or body beneficial properties have not been reported. It would thus be advantageous to prepare niacinamide ascorbate or chitosan niacinamide ascorbate in one single chemical step from readily available ingredients for their direct formulation in skin beneficial cosmetic preparations.

A number of organic acids are also well known for their skin beneficial properties. U.S. Pat. No. 5,861,432 to Sklar describes the use of glycolic acid in an acne treatment formulation. Glycolic acid has been used in many cosmetic formulations for improved skin appearance. There are two main theories on how glycolic acid works. The first theory proposes that the glycolic acid produces a mild sub clinical irritation which stimulates the epidermis to produce fresh skin, while the second theory proposes that glycolic acid weakens the intercellular bonding of the corneocytes in a manner similar to both water and retinoids. Unfortunately, little objective data regarding the effectiveness of alpha-hydroxy acid has been published thereby leaving the industry to rely on anecdotal information, which is difficult to quantify. It is quite clear that many of the topical cosmetics incorporating glycolic acid or other alpha-hydroxy acids have insufficient concentrations to accomplish their objectives. The human skin is comprised of two principal components, the avascular epidermis and the underlying vascular dermis. The epidermis consists of four layers: the stratum corneum, stratum granulosum, stratum spinosum and stratum basale. The dermis mainly consists of collagen, elastin fibers and ground substances including glycosaminoglycan. There are two forms of skin aging: intrinsic aging, also known as chronological aging and extrinsic aging, also known as photo aging. The aging process normally involves the dermis.

Intrinsic aging is a degenerative process attributed to declining physiologic functions and capacities. Extrinsic aging is caused by external factors such as sunlight, radiation, air pollution, etc. AHA's have been used topically in the prior art on keratinization (epidermal layer) where the effects are clinically detectable by the formation of a new stratum corneum. AHA's also have dermal effects. Topical application of AHA's have caused increased amounts of mucopolysaccharides and collagen and increased skin thickness without detectable inflammation. The benefits of the AHA have caused them to be incorporated into cosmetic products for purposes such as cleansing, conditioning, dry skin etc. AHA's are categorized as nontoxic and have been used as skin desquamative agents, especially in routine use for acne, wrinkles, photo aged skin and pigmented disorders. Mandelic acid, another AHA (alpha-hydroxyacid) has been claimed by Yu et al. (U.S. Pat. Nos. 5,677,339 and 5,654,336) in a topical composition for skin wrinkles reduction. Glycolic and lactic acids have been claimed in pimples and skin redness reduction compositions by Slavtcheff et al. (U.S. Pat. Nos. 5,614,201 and 5,482,710). Alliger (U.S. Pat. No. 5,516,799) describe the use of glycolic acid for treating small mouth ulcers. Shaffer et al. (U.S. Pat. No. 5,760,079) describe hydroxy acids for treating striae distensae (stretch marks). Perricone (U.S. Pat. No. 6,417,226) has claimed Hydroxytetronic acid in a skin whitening composition. Other AHA's have shown skin-whitening effects, as mentioned by Zhang et al. (U.S. Patent Application 20020106384).

There is no doubt that alpha hydroxy acids, alpha ketoacids and related compounds are therapeutically effective for topical treatment of various cosmetic conditions and dermatological disorders including dry skin, acne, dandruff, keratoses, age spots, wrinkles and disturbed keratinization. However, the compositions containing these acids may irritate human skin on repeated topical applications due to lower pH of the formulations. The irritation may range from a sensation of tingling, itching and burning to clinical signs of redness and peeling. Causes for such irritation may arise from the following: Upper layers of normal skin have a pH of 4.2 to 5.6, but the compositions containing most alpha hydroxy acids or alpha ketoacids have pH values of less than 3.0. For example, a topical formulation containing 7.6% (1M) glycolic acid has a pH of 1.9, and a composition containing 9% (1M) lactic acid has the same pH of 1.9. These compositions of lower pH on repeated topical applications can cause a drastic pH decrease in the stratum corneum of human skin, and provoke disturbances in intercorneocyte bondings resulting in adverse skin reactions, especially to some individuals with sensitive skin. Moreover, with today's state of the art it is still very difficult to formulate a lotion, cream or ointment emulsion which contains a free acid form of the alpha hydroxyacid, and which is physically stable as a commercial product for cosmetic or pharmaceutical use.

When a formulation containing an alpha hydroxyacid or alpha ketoacid is reacted equimolarly or equinormally with a metallic alkali such as sodium hydroxide or potassium hydroxide the composition becomes therapeutically ineffective. The reasons for such loss of therapeutic effects are believed to be as follows: The intact skin of humans is a very effective barrier to many natural and synthetic substances. Cosmetic and pharmaceutical agents may be pharmacologically effective by oral or other systematic administration, but many of them are much less or totally ineffective on topical application to the skin. Topical effectiveness of a pharmaceutical agent depends on two major factors; (a) bioavailability of the active ingredient in the topical preparation and (b) percutaneous absorption, penetration and distribution of the active ingredient to the target site in the skin. For example, a topical preparation containing 5% salicylic acid is therapeutically effective as a keratolytic, but that containing 5% sodium salicylate is not an effective product. The reason for such difference is that salicylic acid is in bioavailable form and can penetrate the stratum corneum, but sodium salicylate is not in bioavailable form for this specific skin beneficial function and cannot penetrate the stratum corneum of the skin. In the case of alpha hydroxy acids, a topical preparation containing 5% glycolic acid is therapeutically effective for dry skin, but that containing 5% sodium glycolate is not effective. The same is true in case of 5% lactic acid versus 5% sodium lactate. The reason for such difference is that both glycolic acid and lactic acid are in bioavailable forms and can readily penetrate the stratum corneum, but sodium glycolate and sodium lactate are not in bioavailable forms for the intended specific skin beneficial functions and cannot penetrate the stratum corneum of the skin. When a formulation containing an alpha hydroxyacid or alpha ketoacid is reacted equimolarly or equinormally with ammonium hydroxide or an organic base of smaller molecule the composition still shows some therapeutic effects for certain cosmetic conditions such as dry skin, but the composition has lost most of its potency for other dermatological disorders such as wrinkles, keratoses, age spots and skin changes associated with aging. It would thus be surprising if derivatives of such hydroxy acids with an organic base, such as niacinamide, niacin, or niacin ester(s) could offer the above skin beneficial properties of such hydroxy acids despite the most desirable higher pH of such derivatives.

The combination of niacinamide, niacin, or niacin ester(s) with such organic acids to form niacinamide, niacin, or niacin ester(s) derivatives of such organic acid in a simple one step in-situ process is not known in the prior art. Surprisingly, such derivatives of niacinamide, niacin, and niacin ester(s) with organic acids have been found to possess beneficial properties in the present invention that includes synergistic treatment and regulation of topical disorders of skin such as skin aging, wrinkles, acne, rosacea, age-spots, canker sores, striae distensae (stretch marks), pimples, and skin redness. Moreover, the pH of the formulation is not too low or too high for skin's compatibility. Most surprisingly, the AHA's or salicylic acid, in combination with niacinamide, niacin, or niacin ester(s) have a more skin compatible pH and they have not been rendered ineffective, contrary to as mentioned above for the AHA's and salicylic acid that have been neutralized with alkali metal oxides, hydroxides, or ammonium hydroxide to increase their pH.

Among other skin beneficial organic acids, Perricone (U.S. Pat. No. 5,965,618) reports the treatment of scar tissues with lipoic acid. U.S. Pat. No. 6,365,623 to Perricone describes the use of lipoic acid in the treatment of acne. It would thus be advantageous to prepare niacinamide lipoate that could have synergistic combination of skin scar reduction and anti-acne benefits of both niacinamide and lipoic acid. The preparation and applications of such combination for skin treatments is not known in the prior art. Lipoic acid has additional benefits, for example in the treatment of cancer (U.S. Pat. No. 6,284,786 to Casciari et al.), as an antioxidant (U.S. Pat. No. 6,365,622 to Cavazza), in the treatment of central nervous system injuries (U.S. Pat. No. 6,469,049 to Meyerhoff, et al.), and protection against solar radiation (U.S. Pat. No. 6,254,898 to Bragaglia). The enhancement of lipoic acid bioavailability by the present invention can be additionally helpful for the above benefits.

Exposure of skin to ultraviolet (or ionizing) radiation damages DNA, which if unrepaired or improperly repaired, can lead to carcinogenesis as well as contribute to acceleration of the aging process. DNA damage and consequent genomic instability are defining characteristics of both carcinogenesis and biological aging. Patients with defective DNA repair capabilities in diseases like xeroderma pigmentosa display premature skin aging and a very high incidence of skin cancers (Robbins and Moshell, J. Inv. Dermatol., 73:102-107, 1979) on sun-exposed areas of the skin. Pharmacological intervention in damage to skin due to solar or ultraviolet radiation has heretofore been largely restricted to agents like sunscreens or free-radical scavengers intended to prevent damage, or agents like retinoic acid or glycolic acid which are intended to remodel the surface of radiation-damaged skin without necessarily addressing the most fundamental mechanisms of cell or tissue damage and repair at the level of genomic integrity. Von Borstel et al. (U.S. Pat. No. 6,465,440) have discussed in detail the utility of nucleotides and nucleic acids in the treatment of various skin disorders. Additionally, U.S. Pat. No. 5,246,708 discloses the methods and compositions involving the use of mixtures of deoxyribonucleosides for promotion of the healing of wounds, ulcers, and bums, including those caused by ultraviolet or solar radiation. Acyl derivatives of deoxyribonucleosides have been taught as delivery molecules for promoting entry of deoxyribonucleosides into the skin, as disclosed in U.S. Pat. No. 6,297,272. It is disclosed that acyl derivatives of deoxyribonucleosides can improve cellular repair and cell survival after damage to skin caused by radiation.

Since nucleotides and nucleic acids have a phosphate acid group attached to their molecules, it would thus be possible to prepare niacinamide derivatives of such nuleosides and nucleic acids for their synergistic benefit to solve skin disorders, photo-damage to skin and skin aging problems. Such derivatives of niacinamide, niacin, and niacin esters with nucleotides and nucleic acids have not been reported in the prior art, especially by a one step in-situ method from readily available ingredients.

I have discovered a simple method by which derivatives of niacinamide, niacin, or niacin esters (nicotinic acid esters) that can be made in-situ for their inclusion in cosmetic or pharmaceutical compositions that are useful for the synergistic treatment and regulation of topical disorders of skin aging, wrinkles, acne, rosacea, age-spots, canker sores, striae distensae (stretch marks), pimples, and skin redness. The in-situ method comprises the mixing of niacinamide, niacin, or niacin esters with a suitable skin beneficial organic acid in equimolar amounts in a water or water-miscible organic solvent solution. The pH of such solutions, if formulated in compositions that contain water, is adjusted that assures the derivatization (complex formation) of niacinamide, niacin, or niacin esters with the organic acid is complete. Any pH that is too high or too low from the optimum pH range can disrupt the complexation of niacinamide, niacin, or niacin esters with the organic acid. The optimum pH range is specific for each specific complex. The optimum pH range for each specific niacinamide or niacin derivative of an organic acid is determined by first preparing such derivative by an in-situ method in water or in a mixture of water and water-miscible organic solvent, and then determining the pH of such derivative before the incorporation of such derivative in a topical formulation or composition. Surprisingly, such derivatives of niacinamide and niacin are also stable in the presence of water or a mixture of water and water-miscible organic solvent in a solubilized state, and they additionally offer enhanced bioavailability due to their rapid absorption into the skin from such solubilized compositions.

However, the pH of the composition is not important for the preparation of any anhydrous delivery systems. This is due to the fact that niacinamide, niacin, or niacin ester derivatives of organic acids do not usually ionize in such anhydrous systems.

To illustrate the scope of this invention, the equation 1 shows the formation of niacinamide salicylate, a derivative of niacinamide with salicylic acid (an organic acid with skin beneficial anti-acne property), in water solution;

Niacinamide+Salicylic Acid=Niacinamide Salicylate (Equation 1)

Similarly, by mixing niacin with lactic acid in equimolar amounts in water solution, one mole of niacin lactate is produced in-situ, as illustrated in Equation 2.

Niacin+Lactic Acid=Niacin Lactate (Equation 2)

Additionally, by mixing an inorganic acid salt of niacinamide or niacin with a metal salt of an organic acid, niacinamide or niacin derivatives of organic acids can be prepared in-situ, as depicted in Equation 3.

Niacin hydrochloride+Sodium Glycolate=Niacin Glycolate+Sodium Chloride (Equation 3).

Multi-component niacin or niacinamide derivatives of organic acids can also be made by the in-situ method by mixing the reacting components in proportionate molar quantities in water solution, as illustrated in Equation 4.

$$\begin{matrix}Niacinamide + Gylcolic Acid + \\ Hydroxycitric Acid + Ascorbic Acid + \\ Salicylic Acid = Niacinamide Glycolate + \\ Niacinamide Hydroxycitrate + \\ Niacinamide Ascorbate + Niacinamide Salicylate\end{matrix} \qquad ( Equation 4 )$$

Novel derivatives of niacinamide or niacin with skin beneficial organic acids can be made by in-situ method of present invention, as illustrated for the preparation of Niacin Ascorbyl Phosphate in Equation 5.

Sodium Ascorbyl Phosphate+Niacin Hydrochloride=Niacin Ascorbyl Phosphate+Sodium Chloride (Equation 5)

Multi-component compositions of both previously unknown derivatives of niacinamide or niacin and previously known derivatives of niacinamide or niacin can also be made, as illustrated in Equation 6.

$$\begin{matrix}Niacinamide + Niacin Hydrochloride + \\ Sodium Ascorbyl Phosphate + Lactic Acid + \\ Lipoic Acid + Folic Acid = \\ Niacin Ascorbyl Phosphate + Niacinamide Lactate + \\ Niacinamide Lipoate + Niacinamide Folate + \\ Sodium Chloride\end{matrix} \qquad ( Equation 6 )$$

The derivatives of niacin esters with skin beneficial organic acids can be made by the in-situ method by simply mixing the commonly available niacin ester and organic acid ingredients, as illustrated in Equation 7 and Equation 8.

Benzyl Nicotinate+Adenosine monophosphate=Benzyl nicotinate adenosine phosphate (Equation 7)

Methyl Nicotinate+Lipoic Acid=Methyl nicotinate lipoate (Equation 8)

The compositions in Equation 1 to 8 can also be made in anhydrous systems by using appropriate water-soluble organic solvent in place of water in the in-situ method. The water-miscible organic solvents include but not limited to glycerin, propylene glycol, butylene glycol, polyethylene glycol, polypropylene glycol, methyl pyrrolidone, pyrrolidone, butylene glycol, hexylene glycol, methylpropanediol, glycol ethers, ethanol, isopropanol, and such..

The examples shown in Equation 1 to Equation 8 are only illustrative, and they do not represent any limitations of the scope of present invention.

The amount of skin beneficial niacinamide, niacin, or niacin ester derivatives of organic in the formulation is from about 0.1% to about 50% by weight, preferably from 5% to 20% by weight, most preferably from 1 % to 10% by weight. A particular advantage of the current invention is that relatively large amounts of niacinamide, niacin, and niacin ester derivatives, up to about 50% by weight, can be incorporated in the formulation. If the amount of such derivatives is in excess of 50%, the crystallization may become a problem. However, the derivatives of niacinamide, niacin, or niacin esters with organic acids can be made in certain organic solvents in amounts higher than 50%. The skin care benefits of such compositions in such high concentrations are not known at this time, and thus not claimed in the present invention.

The amount of water in the formulation is from about 0% to about 90%. This is because the compositions that contain derivatives of niacinamide, niacin, or niacin esters of the present invention can be made in traditional water and oil emulsions, suspensions, colloids, solutions, or anhydrous systems. For anhydrous systems, the water is typically much less than 1%. The present invention thus permits the formulation of a wide variety of compositions that can contain water or be anhydrous systems. Anhydrous systems may be preferred for certain applications, such as the preparation of high potency facial serums and skin whitening lotions, as will become clearer in the Examples section of this invention, whereas water and oil emulsions and suspensions are typically preferred for lotion, cream, gel, paste, and such.

The amount of the cosmetically acceptable delivery system in the formulation is from 1% to 80%, preferably from 10% to 50% by weight. The delivery system can comprise a base for lotion, cream, shampoo, serum, gel, salve, paste, spray, collodion, and such. The delivery system can be composed of one or more ingredients to provide skin elegance, skin feel, and enhanced bioavailability attributes popularly desired by the consumers.

The pH of the formulation is from about 3.0 to about 7.5, preferably from about 3.5 to about 5.5. The preferable pH is determined by the optimum stability of the complex that is derived from the combination of niacinamide, niacin, or niacin esters with appropriate skin beneficial organic acid. As an illustration, the pH of the desired compositions in column 4 was determined from combining, in equimolar amounts, niacinamide in column 1, with appropriate organic acid in column 2, to give desired niacinamide derivative in column 3. This preparation was done in-situ in a deionized water solution by mixing 0.01 mole of niacinamide in 50 grams of deionized water and then determining the pH of the resulting solution, them preparing a solution or suspension of 0.01 mole of an organic acid in column 2 in 50 grams of deionized water and determining its pH. The solution of niacinamide in water obtained in column 1 is then combined with the solution or suspension of organic acid in water obtained in column 2, to provide a solution of niacinamide derivative of organic acid in deionized water as per column 3. The pH of niacinamide derivative thus obtained in column 3 is also indicated in column 3. The pH in column 3 was determined to be optimal for any formulations that contained the ingredients in column 3.

| Column 1 | Column 2 | Column 3 |
|---|---|---|
| Niacinamide (pH 6.3) | Ascorbic acid (pH 2.6) | Niacinamide ascorbate (pH 3.7) |
| Niacinamide (pH 6.3) | Salicylic acid (pH 2.5) | Niacinamide salicylate (pH 3.5) |
| Niacinamide (pH 6.3) | Lactic acid (pH 2.3) | Niacinamide lactate (pH 3.6) |
| Niacinamide (pH 6.3) | Mandelic acid (pH 2.2) | Niacinamide mandelate (pH 3.3) |
| Niacinamide (pH 6.3) | Lipoic acid (pH 3.6) | Niacinamide lipoate (pH 4.6) |

If a surfactant is desired, then the amount of surfactant in the formulation is from 1% to 30%, preferably from 10% to 30% by weight. It is possible that the amount of surfactant in the formulation can be up to 40% by weight, but concentrations of surfactant greater than 30% increase the risk that the surfactant may gel out. Less than 10% by weight of surfactant is acceptable, but the foaming properties of the formulation are not as good for certain applications, such as a facial acne cleanser. Examples of surfactants that can be utilized are anionic, amphoteric, nonionic and cationic surfactants. Examples of anionic surfactants include, without limitation, soaps, alkyl sulfates, anionic acyl sarcosinates, methyl acyl taurates, N-acyl glutamates, acyl isethionates, alkyl phosphate esters, ethoxylated alkyl phosphate esters, alkyl sulfosuccinates, trideceth sulfates, protein condensates, mixtures of ethoxylated alkyl sulfates, and the like. Examples of anionic non-soap surfactants are, without limitation, the alkali metal salts of organic sulfate having in their molecular structure an alkyl radical containing from about 8 to about 22 carbon atoms and a sulfonic acid or sulfuric acid ester radical. Examples of Zwitterionic surfactants are, without limitation, derivatives of aliphatic quaternary ammonium, phosphonium, and sulfonium compounds, in which the aliphatic radicals can be straight chain or branched and wherein one of the aliphatic substituents contains from about 8 to 18 carbon atoms and one contains an anionic water-solubilizing group, e.g., carboxyl, sulfonate, sulfate, phosphate, or phosphonate. Examples of amphoteric surfactants are, without limitation, derivatives of aliphatic secondary and tertiary amines in which the aliphatic radical can be straight chain or branched and wherein one of the aliphatic substituents contains from about 8 to about 18 carbon atoms and one contains an anionic water solubilizing group, e.g., carboxyl, sulfonate, sulfate, phosphate, or phosphonate. Examples of cationic surfactants are, without limitation, stearyldimenthylbenzyl ammonium chloride; dodecyltrimethyl ammonium chloride; nonylbenzylethyldimethyl ammonium nitrate; and tetradecylpyridinium bromide. Nonionic surfactants include, without limitation, compounds produced by the condensation of alkylene oxide groups (hydrophilic in nature) with an organic hydrophobic compound, which may be aliphatic or alkyl aromatic in nature, for example, the polyethylene oxide condensates of alkyl phenols.

Additional skin, hair, and body beneficial ingredients, such as other anti-aging ingredients, vitamins, hormones, analgesics, anesthetics, sun screens, skin whiteners, anti-acne agents, anti-bacterial agents, anti-fungal agents, botanical extracts, pharmaceuticals, processing-aids, minerals, plant extracts, concentrates of plant extracts, emollients, moisturizers, skin protectants, humectants, silicones, skin soothing ingredients, colorants, perfumes, and like can be added to the formulation. The quantities of such ingredients can be as needed, and not limited to any specific limits.

It is also common to use rheology modifiers for the control of viscosity and to provide skin feel attributes in cosmetic compositions. A variety of rheology modifiers can be used in the compositions of the present invention. The examples of rheology modifiers include, without limitation, Aristoflex AVC (Ammonium Acryloyldimethyltaurate/VP Copolymer), Structure Plus and Structure XL (Acrylates/Aminoacrylates/C10-30 Alkyl PEG-20 Itaconate Copolymer), Carbomer, Xanthan Gum, Gellan Gum, Gum Arabic, Bentonite, various Clays, Silicas, Fumed Silica, Zeolites, Carbopol ETD 2020 ( Acrylate C10-30 Alkyl Acrylate Crosspolymer), Rheocin (trihydroxystearin), Hydramol PGDS (PEG-90 Diisostearate), C24-28 Alkyl Dimethicone, Behenyl alcohol, and other similar materials.

The teachings of the present invention also permit the preparation of improved pharmaceutical compositions. For example, salicylic acid is a known drug ingredient approved by the FDA (Food & Drug Administration) for the cure of acne in the USA. However, if two such formulations from two different competing manufacturers are each made with, let us say 2% salicylic acid, then the clinical efficacy of these two formulations is expected to be very similar. However, by also using only 0.5% to 1% of niacinamide lipoate, as described in the present invention, in one of these formulations that contains 2% salicylic acid, the clinical efficacy for the cure of acne can now be improved over the other formulation that contains only 2% salicylic acid. Similarly, hydroquinone is a drug approved by the FDA for skin whitening compositions. Again, if two competing products had the same amount of hydroquinone, let us say 2%, then the skin whitening benefit will be expected to be same for these two products. However, by also including only 1% to 2% of niacinamide ascorbate in one of these two formulations the skin whitening properties are significantly enhanced, in comparison to the formulation that contains only hydroquinone. In a yet another example, methyl niacin (methyl nicotinate) is an FDA-approved drug ingredient for the treatment of topical pain (topical analgesic drug). However, methyl nicotinate also causes skin irritation. Ursolic acid is a plant-derived ingredient that has found applications as an anti-inflammatory agent and as a skin-soothing agent. A combination of methyl niacin and ursolic acid, made by the teachings of the present invention, results in the preparation of methyl niacin ursolate, which is still an effective topical pain relief agent but yet it does not cause any skin irritation. These examples should serve to illustrate the wide applicability of the present invention in the development of improved medicaments.

The process of present invention is useful for the preparation of novel derivatives of niacinamide, niacin, and niacin esters. Such examples include but not limited to niacinamide salicylate, niacinamide lipoate, niacinamide mandelate, niacinamide lactate, niacinamide glycolate, niacinamide malate, niacinamide adenosine phosphate, niacinamide adenosine triphosphate, niacinamide ascorbate, niacinamide folate, niacinamide hydroxycitrate, niacinamide hydroxytetronate, niacinamide pantothenate, niacin salicylate, niacin lipoate, niacin mandelate, niacin lactate, niacin glycolate, niacin malate, niacin adenosine phosphate, niacin adenosine triphosphate, niacin ascorbate, niacin folate, niacin hydroxycitrate, niacin pantothenate, niacin hydroxytetronate, benzyl nicotinate lipoate (benzyl niacin lipoate), methyl nicotinate lipoate (methyl niacin lipoate), benzyl niacin ascorbate, methyl niacin ascorbate, benzyl niacin salicylate, methyl niacin salicylate, benzyl niacin pantothenate, methyl niacin pantothenate, benzyl niacin lactate, methyl niacin lactate, benzyl niacin malate, methyl niacin malate, lauryl niacin lipoate, lauryl niacin ascorbate, lauryl niacin salicylate, lauryl niacin lactate, methyl niacin glycyrrhetinate, niacinamide glycyrrhetinate, niacinamide glycyrrhizinate, niacinamide hyaluronate, niacinamide pyrrolidone carboxylate, benzyl niacin hyaluronate, benzyl niacin pyrrolidone carboxylate, niacinamide hydroquinone carboxylate, niacin hydroquinone carboxylate, methyl niacin hydroquinone carboxylate, benzyl niacin hydroquinone carboxylate, lauryl niacin hydroquinone carboxylate, methyl niacin ursolate, lauryl niacin ursolate, benzyl niacin ursolate, niacinamide ellagate, niacinamide rosmarinate, niacinamide chloroginate, methyl niacin ellagate, methyl niacin chloroginate, lauryl ellagate, lauryl chloginate, lauryl rosmarinate, and methyl niacin rosmarinate.

### EXAMPLES

The following examples are presented to illustrate presently preferred practice thereof. As illustrations they are not intended to limit the scope of the invention.

All concentrations are in weight %.

### Example 1

This example shows the in-situ preparation of niacinamide ascorbate and its in-situ use for the preparation of a facial cleanser composition. Column 1 describes the ingredients as they are used in the formulation. Column 2 describes the final composition resulting from the in-situ formation of niacinamide ascorbate (an example of niacinamide derivative of an organic acid, as per Equation 1).

| Ingredient | Column 1 | Column 2 |
|---|---|---|
| Glycerin (water miscible organic solvent) | to 100 | to 100 |
| Methyl paraben (preservative) | 0.2 | 0.2 |
| Ascorbic Acid | 9.0 | 0.0 |
| Niacinamide (nicotinamide) | 6.0 | 0.0 |
| Niacinamide Ascorbate | 0.0 | 15.0 |
| Deionized Water | 15.0 | 15.0 |
| Phenoxyethanol (preservative) | 0.9 | 0.9 |
| Tauranol I-78-6 (Sodium Cocoyl Isethionate) | 20.0 | 20.0 |
| (surfactant) | | |
| Tauranol ws conc. (Sodium Methyl Cocoyl | 5.0 | 5.0 |
| Taurate) (surfactant) | | |
| Actiplex 2789 (Extract of various plants) | 0.1 | 0.1 |
| Fragrance | 0.5 | 0.5 |

Procedure: Mix deionized water, ascorbic acid, and niacin in a tank separately. A clear solution is obtained. All of the other ingredients are then added, and the mixture is heated and stirred at 60 to 70 degrees C for about five to ten minutes until the mixture is homogenous. The homogeneous mixture is cooled to room temperature. A paste-like product is formed. The stabilized niacinamide ascorbate formulation is used as a facial, hair, and body cleanser. It should be noted that when the composition is first mixed, as shown in Column 1, it is white in color. After preparation of the batch is complete, the product turns bright yellow, indicating the formation of niacinamide ascorbate, which is naturally yellow in color. The color meter readings were L 91.94, a −7.21, b 22.20.

### Example 2

The paste of Example I is stored at room temperature in a sealed container in the presence of air. After six months the paste is still yellow. A colorimetric reading with a color meter, such as Hunter Color Meter, shows that the color reading has changed by only 5%, and the product is still stable, and has not separated into solid and liquid phases. The color meter readings were L 91.43, a −7.03, b 24.46.

### Example 3

### Anti-Wrinkle Face Gel with In-Situ Preparation of Niacinamide Ascorbate

| Ingredients | Column 1 | Column 2 |
|---|---|---|
| PEG-6 | to 100 | to 100 |
| Ammonium Acryloylmethyltaurate | 1.0 | 1.0 |
| Glycerin Usp | 5.0 | 5.0 |
| Deionized Water | 20.0 | 20.0 |
| Geogard 221 (Preservative) | 0.5 | 0.5 |
| Vitamin E Acetate | 2.0 | 2.0 |
| Niacinamide | 6.0 | 0 |
| Ascorbic Acid | 9.0 | 0 |
| Dimethicone | 4.0 | 4.0 |
| Dimethiconol | 4.0 | 4.0 |
| Cetyl Dimethiconol | 2.0 | 2.0 |
| Niacinamide Ascorbate | 0.0 | 15.0 |

Procedure: All ingredients in Column 1 were mixed and heated at 40 to 50C. for 30 minutes. The mixture was cooled to room temperature. A clear gel was obtained, with analysis reported in Column 2.

### Example 4

### The In-Situ Preparation of a 32.8% High Potency Niacinamide Lipoate Antiage Serum from Niacinamide and Lipoic Acid

| Ingredients | Column 1 | Column 2 |
|---|---|---|
| Lipoic Acid | 20.6 | 0.0 |
| Niacinamide | 12.2 | 0.0 |
| Deionized Water | 33.2 | 33.2 |
| Propylene Glycol | 34.0 | 34.0 |
| Niacinamide Lipoate | 0.0 | 32.8 |

Procedure: All ingredients in Column 1 were mixed and heated at 40 to 50C. for 30 minutes. The product was cooled. A thin solution of composition in Column 2 was obtained.

### Example 5

### The In-Situ Preparation of a 42.9% High Potency Anti-aging and Skin Whitening Niacinamide Glutathione Composition is Described in this Example

| Ingredients | Column 1 | Column 2 |
|---|---|---|
| Glutathione | 30.7 | 0.0 |
| Niacinamide | 12.2 | 0.0 |
| Deionized Water | 50.0 | 50.0 |
| Methylpropanediol | 7.1 | 7.1 |
| Niacinamide Glutathione | 0.0 | 42.9 |

Procedure: All ingredients in Column 1 were mixed. A solution of Niacinamide Glutathione was obtained, as shown in Column 2.

### Example 9

### Preparation of an Eye, face, and Neck Gel with Niacinamide Salicylate, Niacinamide Ascorbate, and Other Skin Beneficial Ingredients

| Ingredient | Column 1 | Column 2 |
|---|---|---|
| PEG-6 | to 100 | to 100 |
| Aristoflex AVC | 1.00 | 1.00 |
| Glycerin USP | 5.00 | 5.00 |
| Deionized Water | 20.00 | 20.00 |
| Geogard 221 (preservative) | 0.50 | 0.50 |
| Vitamin E Acetate | 0.50 | 0.5 |
| Niacinamide | 2.44 | 0.00 |
| Ascorbic Acid | 1.76 | 0.00 |
| Salicylic Acid | 1.38 | 0.0 |
| Dimethicone | 4.00 | 4.00 |
| Dimethiconol | 4.00 | 4.00 |
| Cetyl Dimethiconol | 2.00 | 2.00 |
| Kaempferia Galanga Extract | 0.2 | 0.2 |
| Esculin | 0.5 | 0.5 |
| Boswellia Serrata Extract | 0.2 | 0.2 |
| Methylsulfonylmethane (MSM) | 0.5 | 0.5 |
| Niacinamide Ascorbate | 0.0 | 2.98 |
| Niacinamide Salicylate | 0.0 | 2.6 |

Procedure: Mix all ingredients in Column 1 and heat at 60 to 70C. for 30 minutes. Cool to room temperature, and adjust pH to 4.5 with sodium hydroxide solution. A clear pale yellow gel of composition in Column 2 was obtained.

### Example 10

### Acne Treatment Gel

This example shows the in-situ preparation of an anti-acne, anti-aging, anti-wrinkle composition that contains multi-component mixture of several niacinamide and organic acid complexes. Column 1 describes the ingredients as they are used in the formulation. Column 2 describes the final composition resulting from the in-situ formation of a total of 13.8% of niacinamide - organic acid complexes.

| Ingredient | Column 1 | Column 2 |
|---|---|---|
| Glycerine | to 100 | to 100 |
| Niacinamide | 10.0 | 3.9 |
| Deionized Water | 20.0 | 20.0 |
| Geogard 221 (Preservative) | 0.5 | 0.5 |
| Dow Corning 2501 Wax | 10.0 | 10.00 |
| Structure Plus | 4.0 | 4.00 |
| Eyebright Extract | 0.1 | 0.1 |
| Botanicals Extracts Blend | 0.1 | 0.1 |
| Vitamin E Acetate | 0.1 | 0.1 |
| Lactic Acid | 0.9 | 0.0 |
| N-Acetyl-Cysteine | 1.63 | 0.0 |
| Ascorbic Acid | 1.76 | 0.0 |
| Salicylic Acid | 1.38 | 0.0 |
| Lipoic Acid | 2.06 | 0.0 |
| Niacinamide Lactate | 0.0 | 2.12 |
| Niacinamide N-Acetyl-Cysteinate | 0.0 | 2.85 |
| Niacinamide Ascorbate | 0.0 | 2.98 |
| Niacinamide Salicylate | 0.0 | 2.6 |
| Niacinamide Lipoate | 0.0 | 3.28 |

Procedure: All the ingredients in column 1 were mixed and heated at 60 to 70C. for 30 minutes. A yellow mixture was obtained. The composition of this mixture is shown in Column 2. The product is Column 2 was obtained as a light yellow gel, useful for face and neck zone anti-acne, anti-age and anti-wrinkle applications. The product has the following properties: pH 4.5.

### Example 11

This example illustrates the preparation of a skin whitening formula that contains hydroquinone as the drug active ingredient, with niacinamide ascorbate and niacinamide lactate also added to boost the skin-whitening efficacy. Column 1 shows the ingredients as they are added in the formulation. Column 2 shows the final composition of the formulation.

| | Column 1 | Column 2 |
|---|---|---|
| 1. Carbowax 300 (PEG-6) | To 100 | to 100 |
| 2. Aristoflex AVC | 0.8 | 0.8 |
| 3. Deionized Water | 15.0 | 15.0 |
| 4. Ascorbic Acid | 6.0 | 0.0 |
| 5. Niacinamide | 6.44 | 0.0 |
| 5. Hydroquinone | 4.0 | 4.0 |
| 6. Jeechem HPIB (silicone blend) | 10.0 | 10.0 |
| 7. Killitol (preservative) | 0.3 | 0.3 |
| 8. Lactic Acid | 1.8 | 0.0 |
| 9. Niacinamide Ascorbate | 0.0 | 10.0 |
| 10. Niacinamide lactate | 0.0 | 4.24 |

Process: Mix 2 and 3 till a clear gel is formed. Add 1, 7, and 8 and heat at 50 to 60C. Add all other ingredients with mixing. Cool to room temperature. A translucent cream is obtained.

### Example 12

This example shows in-situ preparation of benzyl niacin ascorbate, benzyl niacin lactate, and benzyl niacin lipoate and their formulation with an oil-soluble vitamin for an anti-acne, skin whitening, and anti-wrinkle composition. Column 1 shows the ingredients as they are added. Column 2 shows the final composition of the formulation.

| Ingredients | Column 1 | Column 2 |
|---|---|---|
| 1. Carbowax 300 | to 100 | to 100 |
| 2. Aristoflex AVC | 1.0 | 1.0 |
| 3. Glycerin | 5.0 | 5.0 |
| 4. Deionized Water | 20.0 | 20.0 |
| 5. Vitamin E Acetate | 2.1 | 2.1 |
| 6. Geogard 221 (Preservative) | 0.5 | 0.5 |
| 7. Dimethicone | 4.0 | 4.0 |
| 8. Benzyl Niacin | 6.39 | 0.0 |
| 9. Ascorbic Acid | 1.76 | 0.0 |
| 10. Lactic Acid | 0.9 | 0.0 |
| 11. Lipoic Acid | 2.06 | 0.0 |
| 12. Dimethiconol | 4.0 | 4.0 |
| 13. Cetyl Dimethicone Copolyol | 2.0 | 2.0 |
| 14. Benzyl Niacin Ascorbate | 0.0 | 3.89 |
| 15. Benzyl Niacin Lactate | 0.0 | 3.03 |
| 16. Benzyl Niacin Lipoate | 0.0 | 4.19 |

Process:

A. Mix 2, 3, and 4 till a clear gel is formed.

B. Add all other ingredients and mix.

C. A translucent cream is obtained.

D. It is rapidly absorbed upon application to skin.

E. No skin flushing is observed. (Skin flushing is observed when only benzyl niacin is applied to skin.)

### Example 13

This example illustrates the preparation of an anhydrous 45.4% serum of methyl niacin lactate for a very high potency skin rejuvenating serum. Column 1 shows the ingredients as they are used in the formulation. Column 2 shows the final composition of the formulation.

| Ingredients | Column 1 | Column 2 |
|---|---|---|
| 1. Carbowax (PEG-6) | to 100 | to 100 |
| 2. Methyl Niacin (methyl nicotinate) | 27.4 | 0.0 |
| 3. Lactic Acid | 18.0 | 0.0 |
| 4. Methyl Niacin Lactate | 0.0 | 45.4 |

Process: Mix all ingredients till a clear solution is obtained. The pH of this product is not important, as it does not contain any water (anhydrous composition).

### Example 14

This example illustrates the preparation of a facial acne treatment cream that contains salicylic acid as a drug active ingredient with the enhancement of its efficacy by the inclusion of niacinamide salicylate and niacinamide lipoate prepared by the in-situ method of the present invention.

| Ingredients | Column 1 | Column 2 |
|---|---|---|
| 1. Polyethylene glycol (PEG-6) | to 100 | to 100 |
| 2. Aristoflex AVC | 1.0 | 1.0 |
| (ammonium acryloyldimethyl- | | |
| taurate/vp copolymer) | | |
| 3. Deionized water | 15.0 | 15.0 |
| 4. Salicylic Acid | 3.38 | 2.0 |
| 5. Lipoic Acid | 2.06 | 0.0 |
| 6. Niacinamide | 2.44 | 0.0 |
| 7. Killitol (preservative) | 0.3 | 0.3 |
| 8. Jeesilc 6056 (polydimethylsiloxane) | 10.0 | 10.0 |
| 9. Actiplex (plant extracts blend) | 0.1 | 0.1 |
| 10. Titanium dioxide | 0.2 | 0.2 |
| 11. Niacinamide Salicylate | 0.0 | 2.6 |
| 12. Niacinamide Lipoate | 0.0 | 3.28 |

Procedure: mix 2 and 3 till clear. Add all other ingredients except 9. Heat at 50 to 60 C. for 30 minutes till all solids dissolved. Cool to room temperature with slow mixing. Add 9. Mix till homogeneous. A white cream is obtained.

I claim:

1. A synergistic cosmetic or pharmaceutical composition for the treatment or prevention of topical disorders of skin such as acne, rosacea, skin wrinkles, age-spots, canker sores, striae distensae (stretch marks), pimples, and redness comprising:
(i) a skin beneficial agent ranging from about 0.1% to about 50%, selected from a derivative of niacinamide with at least one organic acid,
(ii) from about 1% to about 90% of water,
(iii) from about 1% to about 99% of a cosmetically acceptable delivery system, and,
(iv) the pH of the composition from about 3.0 to about 7.5.

2. A composition according to claim 1 wherein the skin beneficial organic acid is selected from the group consisting of hydroxy acids, aromatic acids, fatty acids, vitamin acids, amino acids, amino esters, hydroquinone derivatives, peptides, peptide esters, N-protected amino acids, and combinations thereof.

3. A composition according to claim 1 wherein the skin beneficial organic acid is selected from the group consisting of salicylic acid, lactic acid, glycolic acid, malic acid, mandelic acid, ascorbic acid, ascorbyl phosphoric acid, hydroxycitric acid, hydroxytetronic acid, lipoic acid, folic acid, citric acid, aleuritic acid, ellagic acid, rosmarinic acid, chlorogenic acid, petroselinic acid, pantothenic acid, triterpene acids, steroidal acids, hyaluronic acid (HYA), pyrrolidone carboxylic acid (PCA), glycyrrhetinic acid, glycyrrhizinic acid, glutamic acid, aspartic acid, N-acetyl cystine, N-acetyl cystein, carnosine, glutathione, nucleic acids, nucleotides and combinations thereof.

4. A composition according to claim 1 wherein the skin beneficial agent is selected from the group consisting of niacinamide salicylate, niacinamide ascorbate, niacinamide folate, niacinamide lipoate, niacinamide lactate, niacinamide glycolate, niacinamide mandalate, niacinamide malate, niacinamide hydroxycitrate, niacinamide hydroxytetronate, niacinamide aleurate, niacinamide petroselinate, niacinamide pantothenate, niacinamide adenosine monophosphate (AMP), niacinamide diphosphate (ADP), niacinamide adenosine triphosphate (ATP), niacinamide hydroquinone carboxylate and combinations thereof.

5. A composition according to claim 1 wherein the skin beneficial agent is prepared by an in-situ method.

6. A composition according to claim 1 wherein the cosmetically acceptable delivery system is selected from a lotion, cream, shampoo, shower gel, cleanser, bath oil, salve, paste, lip balm, serum, gel, body splash, cologne, and other such well known topical cosmetic and pharmaceutical delivery systems.

7. The compositions according to claim 1 wherein the cosmetically acceptable delivery system can be traditional water and oil emulsions, suspensions, colloids, or anhydrous systems.

8. A composition according to claim 1 wherein additional skin beneficial ingredients, such as vitamins, hormones, minerals, plant extracts, skin whitening agents, anti-inflammatory agents, concentrates of plant extracts, emollients, moisturizers, skin protectants, humectants, silicones, skin soothing ingredients, sun screens, analgesics, anesthetics, colorants, perfumes, and like can be added to the formulation. The quantities of such ingredients can be as needed, and not limited to any specific limits.

9. A cosmetic or pharmaceutical composition for synergistic treatment or prevention of topical disorders of skin such as acne, rosacea, skin wrinkles, age-spots, canker sores, striae distensae (stretch marks), pimples, and redness comprising:
(i) a skin beneficial agent ranging from about 0.1% to about 50%, selected from a derivative of niacin with at least one organic acid,
(ii) from about 0% to about 90% of water,
(iii) from about 1% to about 99% of a cosmetically acceptable delivery system, and
(iv) the pH of the composition from about 3.0 to about 7.5.

10. A composition according to claim 9 wherein the skin beneficial organic acid is selected from the group consisting of hydroxy acids, aromatic acids, fatty acids, vitamin acids, triterpene acid, steroidal acid, and combinations thereof.

11. A composition according to claim 9 wherein the skin beneficial organic acid is selected from the group consisting of salicylic acid, lactic acid, glycolic acid, malic acid, mandelic acid, ascorbic acid, ascorbyl phosphoric acid, hydroxycitric acid, hydroxy tetronic acid, lipoic acid, folic acid, citric acid, aleuritic acid, ellagic acid, rosmarinic acid, chlorogenic acid, petroselinic acid, pantothenic acid, gluconic acid, hyaluronic acid (HYA), pyrrolidone carboxylic acid (PCA), glycyrrhetinic acid, glycyrrhizic acid, glutamic acid, aspartic acid, N-acetyl cystine, N-acetyl cystein, carnosine, glutathione, nucleic acids, nucleotides and combinations thereof.

12. A composition according to claim 9 wherein the skin beneficial agent is selected from the group consisting of niacin salicylate, niacin ascorbate, niacin folate, niacin lipoate, niacin lactate, niacin glycolate, niacin malate, niacin mandalate, niacin hydroxycitrate, niacin hydroxytetronate, niacin aleurate, niacin petroselinate, niacin pantothenate, niacin adenosine monophosphate (AMP), niacin diphosphate (ADP), niacin adenosine triphosphate (ATP) and combinations thereof.

13. A composition according to claim 9 wherein the cosmetically acceptable delivery system is selected from a lotion, cream, shampoo, shower gel, cleanser, bath oil, salve, paste, lip balm, serum, gel, body splash, cologne, and other such well known topical cosmetic and pharmaceutical delivery systems.

14. The compositions according to claim 9 can be traditional water and oil emulsions, suspensions, colloids, or anhydrous systems.

15. A composition according to claim 9 wherein additional skin beneficial ingredients, such as vitamins, hormones, minerals, plant extracts, concentrates of plant extracts, emollients, moisturizers, skin protectants, humectants, silicones, skin soothing ingredients, sun screens, analgesics, anesthetics, colorants, perfumes, and like can be added to the formulation. The quantities of such ingredients can be as needed, and not limited to any specific limits.

16. A composition according to claim 9 wherein the skin beneficial agent is prepared by an in-situ method.

17. A composition according to claim 9 wherein niacin derivatives prepared by in-situ method are free of flushing effect (a warm feeling in the skin usually associated with redness and itching) normally caused by niacin itself.

18. A cosmetic or pharmaceutical composition for synergistic treatment or prevention of topical disorders of skin such as acne, rosacea, skin wrinkles, age-spots, canker sores, striae distensae (stretch marks), pimples, and redness comprising:
(i) a skin beneficial agent ranging from about 0.1% to about 50%, selected from a derivative of niacin esters with at least one organic acid,
(ii) from about 0% to about 90% of water,
(iii) from about 1% to about 99% of a cosmetically acceptable delivery system, and
(iv) the pH of the composition from about 3.0 to about 7.5.

19. A composition according to claim 18 wherein the skin beneficial organic acid is selected from the group consisting of hydroxy acids, aromatic acids, fatty acids, vitamin acids, and combinations thereof.

20. A composition according to claim 18 wherein the skin beneficial organic acid is selected from the group consisting of salicylic acid, lactic acid, glycolic acid, malic acid, mandelic acid, ascorbic acid, ascorbyl phosphoric acid, hydroxycitric acid, hydroxy tetronic acid, lipoic acid, folic acid, citric acid, aleuritic acid, ellagic acid, rosmarinic acid, chlorogenic acid, petroselinic acid, pantothenic acid, gluconic acid, hyaluronic acid (HYA), pyrrolidone carboxylic acid (PCA), glycyrrhetinic acid, glycyrrhizic acid, nucleic acids, nucleotides and combinations thereof.

21. A composition according to claim 18 wherein niacin ester is selected from the group consisting of alkyl esters, aryl esters, heterocyclic esters, glucosides, and combinations thereof.

22. A composition according to claim 18 wherein niacin ester (nicotinic acid ester) has a carbon chain of from C1 to C22 that can be acyclic, cyclic, or heterocyclic in its structure.

23. A composition according to claim 18 wherein niacin ester is selected from the group consisting of benzyl nicotinate, methyl nicotinate, ethyl nicotinate, phenyl nicotinate, octyl nicotinate, decyl nicotinate, lauryl nicotinate, nicotinic acid glucoside, and combinations thereof.

24. A composition according to claim 18 wherein the cosmetically acceptable delivery system is selected from a lotion, cream, shampoo, shower gel, cleanser, bath oil, salve, paste, lip balm, serum, gel, body splash, cologne, and other such well known topical cosmetic and pharmaceutical delivery systems.

25. A composition according to claim 18 wherein additional skin beneficial ingredients, such as vitamins, hormones, minerals, plant extracts, concentrates of plant extracts, emollients, moisturizers, skin protectants, humectants, silicones, skin soothing ingredients, sun screens, analgesics, anesthetics, colorants, perfumes, and like can be added to the formulation. The quantities of such ingredients can be as needed, and not limited to any specific limits.

26. A composition according to claim 18 wherein the skin beneficial agent is prepared by an in-situ method.

27. A composition according to claim 18 wherein niacin ester derivatives prepared by in-situ method are free of flushing effect (a warm feeling in the skin usually associated with redness and itching) normally caused by niacin itself.

28. The compositions according to claim 18 can be traditional water and oil emulsions, suspensions, colloids, or anhydrous systems.

29. Additional skin beneficial ingredients that can be used according to claim 8 include hydroquinone, arbutin, hydroquinone derivatives, and other such skin whitening agents.
